(19) United States
(12) Patent Application Publication
(10) Pub. No.: US 2024/0343836 A1
(43) Pub. Date: Oct. 17, 2024

(54) PEPTIDE NUCLEIC ACID DEGRADATION COMPLEXES AND USES THEREOF

(71) Applicant: VIRITIS LTD, Petach Tikva (IL)

(72) Inventors: Amit Mery PHILOSOPH, Yehud (IL); Amit TURMAN, Nof-Hagalil (IL); Aaron WISTOUCH, Petach Tikva (IL)

(21) Appl. No.: 18/748,574

(22) Filed: Jun. 20, 2024

Related U.S. Application Data
(63) Continuation of application No. PCT/IL2023/050005, filed on Jan. 1, 2023.

(30) Foreign Application Priority Data
Jan. 2, 2022 (IL) 289578

(51) Int. Cl. C07K 19/00; A61K 31/7088; A61K 38/00; A61P 31/14; C07K 14/00

(52) U.S. Cl. CPC C07K 19/00; A61K 31/7088; A61P 31/14; C07K 14/003; A61K 38/00

(57) ABSTRACT

A composition comprising a peptide nucleic acid (PNA) molecule comprising a nucleotide base sequence configured to bind to a target polynucleotide molecule; and a degron protein covalently bound to the PNA, wherein binding of the PNA to the target polynucleotide molecule causes degron mediated degradation of the target polynucleotide molecule.

### CROSS REFERENCE TO RELATED APPLICATIONS

This application is a Bypass Continuation of PCT Patent Application No. PCT/IL2023/050005 having International filing date of Jan. 1, 2023, which claims the benefit of priority of Israeli Patent Application No. 289578, filed Jan. 2, 2022, the contents of which are all incorporated herein by reference in their entirety.

### REFERENCE TO AN ELECTRONIC SEQUENCE LUSTING

The contents of the electronic sequence listing (VRT 001 PCT sequence listing.xml; Size: 19,267 bytes; and Date of Creation: Jun. 20, 2024) is herein incorporated by reference in its entirety.

### TECHNICAL FIELD OF INVENTION

This disclosure generally relates to peptide nucleic acid (PNA) molecules comprising a nucleotide base sequence configured to bind to a target polynucleotide molecule; and a degron protein covalently bound to the PNA, wherein binding of the PNA to the target polynucleotide molecule causes degron mediated degradation of the target polynucleotide molecule.

### BACKGROUND

PNAs are synthetic DNA-like molecules in which the phosphodiester backbone is replaced by repetitive units of N-(2-aminoethyl) glycine to which the purine and pyrimidine bases are attached via a methyl carbonyl linker. PNA is designed to space out the bases that it carries at the same distances as found in genuine nucleic acids. This enables a strand of PNA to base pair with a complementary strand of DNA or RNA.

Unlike DNA and RNA, the PNA backbone is not charged. Consequently, there is no electrostatic repulsion when PNAs hybridize to its target nucleic acid sequence, giving a higher stability to the PNA-DNA or PNA-RNA duplexes than the natural homo- or heteroduplexes.

PNA has the capability to hybridize with high affinity and sequence specificity to complementary DNA or RNA sequence, obeying the Watson-Crick hydrogen-bonding scheme in which adenine pairs with thymine via two hydrogen bonds and cytosine forms three hydrogen bonds with guanine. In contrast to DNA, PNA can bind in either parallel or antiparallel manner. However, the antiparallel binding is favored over the parallel one.

PNAs can inhibit transcription and translation of genes by tight binding to DNA or mRNA. PNA-mediated inhibition of gene transcription is mainly due to the formation of strand invaded complexes or strand displacement in a DNA target. Since PNAs are not substrates for RNAse, their antisense effect acts through steric interference of either RNA processing, transport into cytoplasm or translation, caused by binding to the mRNA.

The good stability of PNA oligomers, their strong binding efficiency and their lack of toxicity at even relatively high concentrations suggests that PNAs could constitute highly efficient compounds for effective antisense/antigene application. However, despite the initial rapid success of PNA-based approaches in vitro, progress in the use of PNAs as tools for regulating gene expression was hampered by the slow cellular uptake of ‘naked’ PNAs by living cells.

However, due to the absence of a clearing mechanism, PNAs may lead to systemic toxicity. In fact, efforts to improve PNA potency by stabilizing the peptide portion of the drug resulted in toxicity. There thus remains an urgent need to lay attention towards the toxicological aspect of PNA.

Intracellular protein degradation in eukaryotes is mainly achieved by the ubiquitin-proteasome system (UPS). The UPS plays an essential role in maintaining cellular homeostasis by participating in protein quality control, for instance, by recognizing and rapidly degrading incorrectly folded or assembled proteins. In addition to the elimination of aberrant proteins, this system is responsible for the dynamic protein turnover involved in cell regulation. The UPS includes a network of enzymes that targets proteins for degradation. Fine-tuning the abundance of intracellular proteins usually involves E3 ligases that specifically recognize localized sequence elements, called degrons, in their substrates

A degron is defined as the minimal element within a protein that is sufficient for targeting the protein for degradation. Known degrons include short amino acid sequences, structural motifs and exposed amino acids located anywhere in the protein. While there are many types of different degrons, and a high degree of variability even within these groups, degrons are all similar for their involvement in regulating the rate of a protein's degradation.

An important property of degrons is that they are transferable, and the attachment of such sequence elements by means of genetic engineering confers instability on otherwise long-lived proteins.

### SUMMARY

There is provided herein, according to some aspects of the disclosure, a peptide nucleic acid (PNA) molecule comprising a nucleotide base sequence configured to bind to a target polynucleotide molecule; and a degron protein covalently bound to the PNA.

Advantageously, binding of the PNA to the target polynucleotide molecule causes degradation of the target polynucleotide molecule, thereby reducing PNA accumulation in the cells. According to some embodiments, the degradation may be degron mediated degradation. According to some embodiments, the degradation is proteasomal degradation. According to some embodiments, the degradation is lysosomal degradation.

Advantageously, the herein disclosed degron-bound PNA may serve as a platform for non-toxic targeting of molecules of interest, such as, but not limited to viral DNA/RNA, and/or overexpressed endogenous RNAs (e.g. Bc12 and/or Myc overexpression)

The platform includes three parts:
1. A casing—a biological nanobot that enable the molecule to enter a target cell;
2. A body—a hybrid molecule (e.g. PNA-degron or PNA-lysosomal targeting peptide) that has its own mechanism of destruction, optionally in a controllable/inducible manner; and
3. A “warhead”—the nucleobases of the PNA that have strong affinity to a specific target sequence e.g. to a specific viral gene or to a certain oncogene.

It is thus understood that by adapting the sequence of the nucleobases of the PNA, various genes may be targeted thus advantageously making the platform suitable for treating a wide range of diseases (e.g. a wide range of viruses as well as enable the targeting of a same disease (e.g., a same virus) by simultaneous targeting of a number of its (essential) genes.

According to some embodiments, there is provided a composition comprising a peptide nucleic acid (PNA) molecule comprising a nucleotide base sequence configured to bind to a target polynucleotide molecule; and a degron protein covalently bound to the PNA, wherein binding of the PNA to the target polynucleotide molecule causes degron mediated degradation of the target polynucleotide molecule.

According to some embodiments, the target polynucleotide molecule is a DNA or an RNA molecule.

According to some embodiments, the target polynucleotide molecule is a viral polynucleotide molecule. According to some embodiments, the viral polynucleotide molecule is an RNA-dependent RNA polymerase or a spike protein. According to some embodiments, the viral polynucleotide molecule is from corona virus. According to some embodiments, the viral polynucleotide molecule is from a herpes virus.

According to some embodiments, the target polynucleotide molecule is a human polynucleotide molecule overexpressed in cancer. According to some embodiments, the target polynucleotide molecule is a mutated human polynucleotide molecule. According to some embodiments, the human polynucleotide molecule is Bcl-2, c-myc or KRAS. Each possibility is a separate embodiment

According to some embodiments, the composition further comprises a carrier. According to some embodiments, the carrier is an exosome, a liposome, a nanoparticle or any combination thereof. Each possibility is a separate embodiment.

According to some embodiments, the degradation peptide is a degron protein. According to some embodiments, the degron protein comprises the amino acid sequence set forth in SEQ ID NO: 1 or 2. Each possibility is a separate embodiment. According to some embodiments, the degron protein comprises the amino acid sequence set forth in SEQ ID NO: 1.

According to some embodiments, the degradation peptide is a lysosome targeting peptide. According to some embodiments, the lysosome targeting peptide comprises the amino acid sequence set forth in SEQ ID NO: 3 or 4.

According to some embodiments, the nucleobase sequence of the PNA comprises the sequence set forth in SEQ ID NO: 5-15. Each possibility is a separate embodiment.

According to some embodiments, the composition is for use in preventing, ameliorating or treating a viral infection. According to some embodiments, the composition is for use in preventing, ameliorating or treating a cancer in a subject in need thereof.

According to some embodiments, there is provided a method for preventing, ameliorating or treating infection of a subject with a virus, the method comprising administering to the subject a composition comprising a PNA molecule, the PNA molecule comprising: a nucleotide base sequence configured to bind a polynucleotide of the virus; and a degron protein covalently bound to the PNA molecule, wherein binding of the PNA to the viral polynucleotide molecule causes degron mediated degradation of the viral polynucleotide molecule, thereby reducing a load of the virus in the subject.

According to some embodiments, the virus is a positive strand RNA virus. According to some embodiments, the virus is a SARS-CoV2 virus. According to some embodiments, the virus is an influenza virus. According to some embodiments, the virus is a herpes virus.

According to some embodiments, the viral polynucleotide targeted by the PNA is essential to the virus. According to some embodiments, the viral polynucleotide targeted by the PNA molecule is an RNA-dependent RNA polymerase of the virus.

According to some embodiments, there is provided a method for preventing, ameliorating or treating cancer in a subject in need thereof, the method comprising administering to the subject a composition comprising a PNA molecule, the PNA molecule comprising: a nucleotide base sequence configured to bind a polynucleotide molecule of the subject associated with the cancer; and a degron protein covalently bound to the PNA molecule, wherein binding of the PNA molecule to the polynucleotide molecule causes degron mediated degradation of the polynucleotide molecule, thereby reducing expression of the polynucleotide in the subject.

According to some embodiments, the polynucleotide targeted by the PNA molecule is a polynucleotide overexpressed in the cancer. According to some embodiments, the polynucleotide targeted by the PNA molecule is a polynucleotide mutated in the cancer.

Certain embodiments of the present disclosure may include some, all, or none of the above advantages. One or more technical advantages may be readily apparent to those skilled in the art from the figures, descriptions and claims included herein. Moreover, while specific advantages have been enumerated above, various embodiments may include all, some or none of the enumerated advantages.

In addition to the exemplary aspects and embodiments described above, further aspects and embodiments will become apparent by reference to the figures and by study of the following detailed descriptions.

### BRIEF DESCRIPTION OF THE FIGURES

The patent or application file contains at least one drawing executed in color. Copies of this patent or patent application publication with color drawing(s) will be provided by the Office upon request and payment of the necessary fee.

The invention will now be described in relation to certain examples and embodiments with reference to the following illustrative figures so that it may be more fully understood.

FIG. 1 is an illustrative scheme of the herein disclosed PNA-degron platform, according to some embodiment.

FIG. 2 is an illustrative scheme of the herein disclosed inducible—PNA-degron platform, according to some embodiment.

FIG. 3 is an illustrative scheme of the herein disclosed PNA-lysosome-targeting-peptide platform, according to some embodiment.

FIG. 4 shows an HLPC diagram obtained for the synthesized PNA-degron molecule.

FIG. 5 shows flow cytometry analysis of fluorescence intensity obtained 4 hr after transfection with 10 μg/ml PNA-degron (lower panel) as compared to control (upper panel).

FIG. 6 show representative images of florescence images of HEK293 cells transfected with the herein disclosed PNA-degron, 4 h and 24 h post transfection.

FIG. 7 show representative florescence images of HEK293 cells transfected with the herein disclosed PNA-degron and infected with 2 MOI OC43 virus-GFP.

### DETAILED DESCRIPTION

In the following description, various aspects of the disclosure will be described. For the purpose of explanation, specific configurations and details are set forth in order to provide a thorough understanding of the different aspects of the disclosure. However, it will also be apparent to one skilled in the art that the disclosure may be practiced without specific details being presented herein. Furthermore, well-known features may be omitted or simplified in order not to obscure the disclosure.

For convenience, certain terms used in the specification, examples, and appended claims are collected here. Unless otherwise defined, all technical and scientific terms used herein have the same meaning as commonly understood by one of ordinary skill in the art to which this invention pertains.

### Definitions

The articles “a” and “an” are used herein to refer to one or to more than one (i.e., to at least one) of the grammatical object of the article. By way of example, “an element” means one element or more than one element.

Ranges: throughout this disclosure, various aspects of the invention can be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 2.7, 3, 4, 5, 5.3, and 6. This applies regardless of the breadth of the range.

“About” as used herein when referring to a measurable value such as an amount, a temporal duration, and the like, is meant to encompass variations of .+−0.20% or in some instances .+−0.10%, or in some instances .+−0.5%, or in some instances .+−0.1%, or in some instances .+−0.0.1% from the specified value, as such variations are appropriate to perform the disclosed methods.

As used herein, the term essentially devoid of, refers to an ingredient being present in residual amounts only, such as less than 1% w/w, less than 0.5% w/w, less than 0.1% w/w or less than 0.05% w/w of the product.

As used herein, the term “peptide nucleic acid” or “PNA” refer to PNAs are synthetic DNA-like molecules in which the phosphodiester backbone is replaced by repetitive units of N-(2-aminoethyl) glycine to which the purine and pyrimidine bases are attached via a methyl carbonyl linker. Peptide nucleic acids can optionally comprise substitution on the N-(2-aminoethyl)-glycine backbone.

In some embodiments, the C-terminus or N-terminus of the PNA is substituted with a cell-permeabilizing group. In some embodiments, the cell permeabilizing group is a polypeptide comprising 3 to 8 lysine residues. In some embodiments, the polypeptide is linked to the peptide nucleic acid via an amide bond. In some embodiments, the polypeptide is linked to the peptide nucleic acid via a peptide bond, a disulfide bond, or a linker comprising two penicillamine residues bound by a disulfide bond.

In some embodiments, the PNA can comprise a moiety that improves cell-permeability of the compound relative to a PNA without the moiety.

As used herein, the term “nucleobase” refers to naturally occurring and non-naturally occurring heterocyclic moieties known to those who utilize nucleic acid technology or utilize peptide nucleic acid technology to thereby generate polymers that sequence specifically hybridize/bind to nucleic acids by any means, including without limitation through Watson-Crick and/or Hoogsteen binding motifs.

As used herein, the term “degron” refers to a compound that serves to link a targeted protein to a ubiquitin ligase for proteosomal degradation. In eukaryotes, the N-degron comprises at least two determinants: a destabilizing N-terminal residue and a specific internal lysine residue (or residues). The latter is the site of attachment of a multiubiquitin chain, whose formation is required for the degradation of at least some N-end rule substrates. Ubiquitin is a protein whose covalent conjugation to other proteins plays a role in a number of cellular processes, primarily through routes that involve protein degradation.

As used herein, the terms “degron PNA fusion,” or “degron fusion” as used herein refer to a fusion comprising a degron in combination with PNA as a continuous chain, which does not occur in nature.

According to some embodiments, the degron may be substituted with a lysosomal targeting peptide. As used herein, the term “lysosomal degradation peptide” may refer to any peptide driving lysosomal degradation of proteins. Non-limiting examples of suitable lysosomal degradation peptides include an AUTAC peptide (SEQ ID NO: 3) or an ATTEC peptide (SEQ ID NO: 4).

“Operably linked” refers to an arrangement of elements wherein the components so described are configured so as to perform their usual function. For example, a given promoter operably linked to a coding sequence is capable of effecting the expression of the coding sequence when the proper enzymes are present. The promoter need not be contiguous with the coding sequence, so long as it functions to direct the expression thereof. Thus, for example, intervening untranslated yet transcribed sequences can be present between the promoter sequence and the coding sequence and the promoter sequence can still be considered “operably linked” to the coding sequence. In another example, a degron operably linked to a PNA is capable of promoting degradation of the attached PNA-DNA/RNA complex when the proper cellular degradation system (e.g., proteasome or autophagosome degradation) is present. In yet another example, a lysosome targeting peptide operably linked to a PNA is capable of promoting lysosomal degradation of the attached PNA-DNA/RNA complex.

As used herein, the term “polynucleotide” refers to a chain of nucleotides. Furthermore, nucleic acids are polymers of nucleotides. Thus, nucleic acids and polynucleotides as used herein are interchangeable. One skilled in the art has the general knowledge that nucleic acids are polynucleotides, which can be hydrolyzed into the monomeric “nucleotides.” The monomeric nucleotides can be hydrolyzed into nucleosides. As used herein polynucleotides include, but are not limited to, all nucleic acid sequences which are obtained by any means available in the art, including, without limitation, recombinant means, i.e., the cloning of nucleic acid sequences from a recombinant library or a cell genome, using ordinary cloning technology and PCR.TM., and the like, and by synthetic means.

As used herein, the term “encoding” refers to the inherent property of specific sequences of nucleotides in a polynucleotide, such as a gene, a cDNA, or an mRNA, to serve as templates for synthesis of other polymers and macromolecules in biological processes having either a defined sequence of nucleotides (i.e., rRNA, tRNA and mRNA) or a defined sequence of amino acids and the biological properties resulting therefrom. Thus, a gene encodes a protein if transcription and translation of mRNA corresponding to that gene produces the protein in a cell or other biological system. Both the coding strand, the nucleotide sequence of which is identical to the mRNA sequence and is usually provided in sequence listings, and the non-coding strand, used as the template for transcription of a gene or cDNA, can be referred to as encoding the protein or other product of that gene or cDNA.

The phrase “inhibit,” as used herein, means to reduce a molecule, a reaction, an interaction, a gene, an mRNA, and/or a protein's expression, stability, function or activity by a measurable amount or to prevent entirely. Inhibitors are compounds that, e.g., bind to, partially or totally block stimulation, decrease, prevent, delay activation, inactivate, desensitize, or down regulate a protein, a gene, and an mRNA stability, expression, function and activity.

As used herein, the terms “peptide,” “polypeptide,” and “protein” are used interchangeably, and refer to a compound comprised of amino acid residues covalently linked by peptide bonds. A protein or peptide must contain at least two amino acids, and no limitation is placed on the maximum number of amino acids that can comprise a protein's or peptide's sequence. Polypeptides include any peptide or protein comprising two or more amino acids joined to each other by peptide bonds. As used herein, the term refers to both short chains, which also commonly are referred to in the art as peptides, oligopeptides and oligomers, for example, and to longer chains, which generally are referred to in the art as proteins, of which there are many types. “Polypeptides” include, for example, biologically active fragments, substantially homologous polypeptides, oligopeptides, homodimers, heterodimers, variants of polypeptides, modified polypeptides, derivatives, analogs, fusion proteins, among others. The polypeptides include natural peptides, recombinant peptides, synthetic peptides, or a combination thereof.

As used herein, the term “homologous” refers to the sequence similarity or sequence identity between two polypeptides or between two nucleic acid molecules. When a position in both of the two compared sequences is occupied by the same base or amino acid monomer subunit, e.g., if a position in each of two DNA molecules is occupied by adenine, then the molecules are homologous at that position. The percent of homology between two sequences is a function of the number of matching or homologous positions shared by the two sequences divided by the number of positions compared X 100. For example, if 6 of 10 of the positions in two sequences are matched or homologous then the two sequences are 60% homologous. By way of example, the DNA sequences ATTGCC and TATGGC share 50% homology. Generally, a comparison is made when two sequences are aligned to give maximum homology.

As used herein, the term “disorder” in a human subject or a non-human mammal is a state of health in which the subject is able to maintain homeostasis, but in which the subject's state of health is less favorable than it would be in the absence of the disorder. Left untreated, a disorder does not necessarily cause a further decrease in the subject's state of health.

A disease or disorder is “alleviated” if the severity of a sign or symptom of the disease or disorder, the frequency with which such a sign or symptom is experienced by a subject, or both, is reduced.

As used herein, the phrase “therapeutically effective amount,” refers to an amount that is sufficient or effective to prevent or treat (delay or prevent the onset of, prevent the progression of, inhibit, decrease or reverse) a disease or condition, including alleviating symptoms of such diseases.

### EMBODIMENTS

There is provided herein, according to some aspects of the disclosure composition comprising peptide nucleic acid (PNA) molecule comprising a nucleobase sequence configured to bind to a target polynucleotide molecule; and a degron protein and/or degradation sequence (e.g. lysosomal degradation peptide) covalently bound to the PNA.

The PNA has the capability to hybridize with high affinity and sequence specificity to complementary DNA or RNA sequence, such as but not limited to sequences of disease-causing genes, such as viral genes, bacterial genes or endogenous upregulated genes. The PNA inhibits the transcription and/or translation of the target gene by tight binding to DNA or mRNA thereof.

Advantageously, by being covalently bound to a degron or to a lysosomal targeting peptide, the PNA-DNA/RNA complex is degraded thereby avoiding intercellular accumulation, which is particularly important when large concentrations of PNA is required.

According to some embodiments, the degron is a compound that is capable of binding to or binds to a ubiquitin ligase. In further embodiments, the degron is a compound that is capable of binding to or binds to a E3 ubiquitin ligase. The N-degron is an intracellular degradation signal whose essential determinant is a specific (“destabilizing”) N-terminal amino acid residue of a substrate protein. A set of N-degrons containing different destabilizing residues is manifested as the N-end rule, which relates the in vivo half-life of a protein to the identity of its N-terminal residue. The fundamental principles of the N-end rule, and the proteolytic pathway that implements it, are well-established in the literature (see, e.g., Bachmair et al., Science 234: 179 (1986); Varshavsky, Cell 69: 725 (1992)).

In some cases, a suitable degron is constitutive such that the degron exerts its influence on protein stability independent of experimental control.

According to some embodiments, the degron may be an inducible degron. According to some embodiments, the inducible degron bears a destabilizing N-terminal residue in such a way that the N-end rule pathway is only activated under a certain condition resulting. Without being bound by any theory, the inducible condition may activate a previously cryptic N-degron by increasing exposure of a (destabilizing) N-terminal residue, by increasing mobilities of its internal Lys residues, or because of both effects at once. Since proteolysis by the N-end rule pathway is highly processive, any protein of interest can be made short-lived at a first (nonpermissive) condition, but not at a second (permissive) condition.

Examples of suitable inducible degrons include, but are not limited to, those degrons controlled by Shield-1, DHFR, auxins, and/or temperature. Each possibility is a separate embodiment.

According to some embodiments, the degron may include a thermolabile protein bearing a destabilizing N-terminal residue in such a way that the protein becomes a substrate of the N-end rule pathway only at a temperature high enough to result in at least partial unfolding of the protein. Without being bound by any theory, the unfolding activates a previously cryptic N-degron in the protein by increasing exposure of its (destabilizing) N-terminal residue, by increasing mobilities of its internal Lys residues, or because of both effects at once. Since proteolysis by the N-end rule pathway is highly processive, any protein of interest can be made short-lived at a high (nonpermissive) but not at a low (permissive) temperature by expressing it as a fusion to the thus engineered thermolabile protein, with the latter serving as a portable, heat-inducible N-degron module.

According to some embodiments, the degron is a drug controllable degron. According to some embodiments, if the degron is a drug inducible degron, the presence or absence of drug can switch the protein from an “off” (i.e., unstable) state to an “on” (i.e., stable) state or vice versa. An exemplary drug inducible degron is derived from the FKBP12 protein. The stability of the degron is controlled by the presence or absence of a small molecule that binds to the degron.

According to some embodiments, the degron may include the amino acid sequence set forth in SEQ ID NO: 1 (SHGFPPAVAAQDDGTLPMSCAQESGMDRHPAACASARINV). According to some embodiments, the degron may include the amino acid sequence set forth in SEQ ID NO: 2 (ALAPYIP).

According to some embodiments, the degradation sequence may be a lysosome targeting peptide. According to some embodiments, the lysosome targeting peptide may include the amino acid sequence set forth in SEQ ID NO: 3 (AUTAC). According to some embodiments, the lysosome targeting peptide may include the amino acid sequence set forth in SEQ ID NO: 4 (ATTEC).

According to some embodiments, the target polynucleotide molecule is a viral gene. According to some embodiments, the target gene is essential for replication of a virus and or for the formation of virions. Non-limiting examples of genes that may be inhibited include, a viral DNA polymerase, a viral RNA dependent RNA/DNA polymerase, a helicase, a membrane protein, a spike protein, a nucleocapsid protein, an envelope protein. Each possibility is a separate embodiment. According to some embodiments, more than one viral gene may be targeted such as 2, 3, 4, 5 target genes by a respective number of PNAs. Each possibility is a separate embodiment. According to some embodiments, the virus may an RNA virus. According to some embodiments, the virus may be a positive strand RNA virus. According to some embodiments, the virus may be a negative strand RNA virus. According to some embodiments, the virus is Severe acute respiratory syndrome coronavirus 2 (SARS-CoV-2), also known as the coronavirus, which is a positive-sense single-stranded RNA virus.

According to some embodiments, the target polynucleotide molecule may be viral spike-protein gene. According to some embodiments, the target polynucleotide molecule may be the RNA of the spike-protein gene of OC43.

According to some embodiments, the nucleobase sequence of the PNA (targeting (antisense to) the human corona spike protein RNA) may comprise or consist of the sequences set forth in SEQ ID NO: 5 (GCCAATCGCAGATGTTTACCGACGCAAACCT). According to some embodiments, the nucleobase sequence of the PNA (targeting antisense to) the human corona spike protein RNA) may comprise or consist of the sequences set forth in SEQ ID NO: 11 (GCCAATCGCAGATGTTTA).

According to some embodiments, the target polynucleotide molecule may be the spike-protein gene of SARS-CoV-2. According to some embodiments, the nucleobase sequence of the PNA (targeting (antisense to) the SARS-CoV-2 RNA) may comprise or consist of the sequences set forth in SEQ ID NO: 6 (GATCCTTATGAAGATTTTCAAG).

According to some embodiments, the target polynucleotide molecule may be a viral RNA-dependent RNA polymerase gene.

According to some embodiments, the target polynucleotide molecule may be the RNA-dependent RNA polymerase gene of OC43. According to some embodiments, the nucleobase sequence of the PNA (targeting (antisense to) the human corona RNA-dependent RNA polymerase RNA) may comprise or consist of set forth in SEQ ID NO: 7 (TAGCAGATTCTTATTATTCTTATATCATGCCT), SEQ ID NO: 12 (CAGATTCTTATTATTCTTATATCAT) or SEQ ID NO: 13 (CAGATTCTTATTATTCTTAT).

According to some embodiments, the target polynucleotide molecule may be the RNA-dependent RNA polymerase gene of SARS-CoV-2. According to some embodiments, the nucleobase sequence of the PNA (targeting (antisense to the RNA polymerase gene of SARS-CoV-2 RNA) may comprise or consist of the sequences set forth in SEQ ID NO: 8 (ACCTCATCAGGAGATGC).

According to some embodiments, the targeted virus is a herpes virus such as herpes simplex virus type I (HSV-1). According to some embodiments, the target polynucleotide molecule may be a viral DNA polymerase gene. According to some embodiments, the polynucleotide molecule may target UL30 DNA polymerase (UL30) gene of HSV-1. According to some embodiments, the nucleobase sequence of the PNA (targeting (antisense to the DNA polymerase RNA) may comprise or consist of the sequence set forth in SEQ ID NO: 14 (GGACGAGGAGCGGCCAGAGGA). According to some embodiments, the nucleobase sequence of the PNA (targeting (antisense to HSV-1 RNA) may comprise or consist of sequence set forth in SEQ ID NO: 15 (TACATCGGCGTCATCTACGGGGGT).

According to some embodiments, the target polynucleotide molecule may a human polynucleotide molecule overexpressed in cancer, also referred to as oncogenes or proto-oncogenes. Non-limiting examples of suitable such genes include Bcl-2, c-myc, KRAS or any other overexpressed oncogene. According to some embodiments, more than one oncogene may be targeted such as 2, 3, 4, 5 oncogenes genes by a respective number of PNAs. Each possibility is a separate embodiment.

According to some embodiments, the target polynucleotide may be Homo sapiens MYC proto-oncogene, bHLH transcription factor (MYC). According to some embodiments, the nucleobase may have the sequence set forth in SEQ ID NO: 9: ACGAGGAGGAGAACTTCTACCAGCAGCAGC (targeting (antisense to) MYC RNA).

According to some embodiments, the target polynucleotide may be Homo sapiens Bc12. According to some embodiments, the nucleobase may have the sequence set forth in SEQ ID NO: 10: CCTCCCAGAGGAAAA (targeting Bc12).

According to some embodiments, gene inhibition platform further includes a delivery molecule and/or carrier.

According to some embodiments, the delivery may be achieved by utilizing PNA conjugates such as but not limited to GaINAc or cationic cell-penetrating peptides (CPPs).

An alternative to conjugation is the incorporation of the PNA into exosomes, liposomes, lipid nanoparticles, inorganic nanocarriers, or carbon nanocarriers. Each possibility is a separate embodiment.

According to some embodiments, the composition is suitable for use in preventing, ameliorating or treating a viral infection, such as but not limited to SARS-CoV-2 infection.

According to some embodiments, the composition is suitable for use in preventing, ameliorating or treating a cancer in a subject in need thereof.

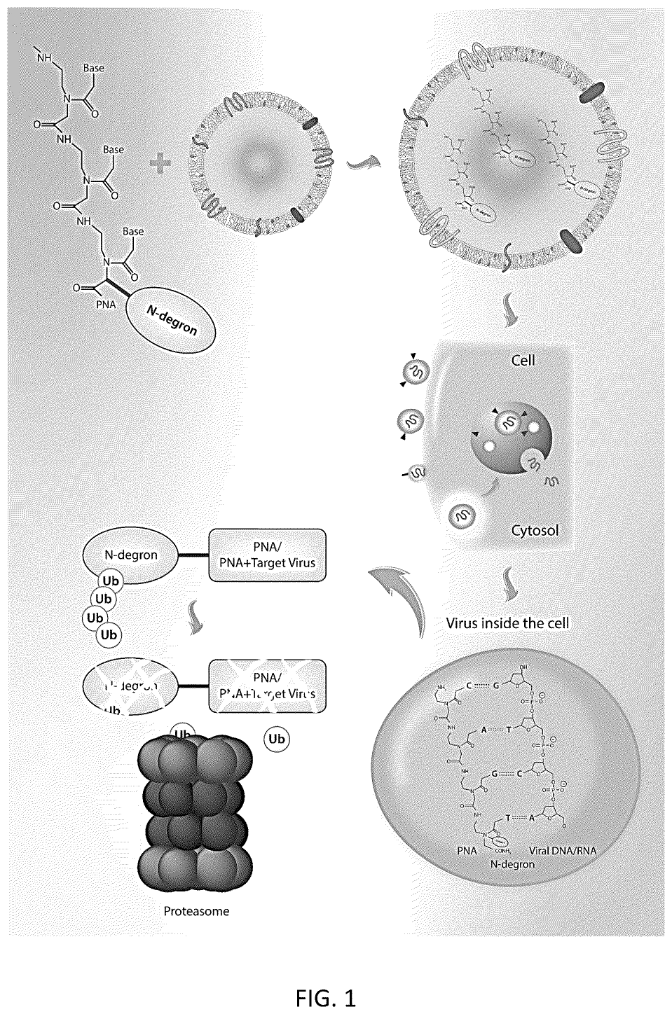

Reference is now made to FIG. 1, which illustratively depicts the operation/use of the herein disclosed PNA-degron platform, according to some embodiment. Initially, a degron bound PNA with a nucleobase sequence targeting a gene of interest a viral RNA/DNA (e.g. RNA dependent RNA polymerase of SARS-CoV-2) is provided and assembled within a carrier, such as, but not limited to an exosome (as illustrated in FIG. 1). The exosome facilitates delivery of the degron bound PNA by way of endocytosis. It is understood that other PNA delivery methods are known in the art and such delivery methods are also envisaged as part of this disclosure.

Inside the cell, the degron bound PNA binds the target gene (here the viral RNA/DNA). Due to the PNA being attached to the degron, the degron-PNA-target gene complex is ubiquitinated which in turn bring about its degradation.

Advantageously, this ensures both inhibition of the target gene (as essentially described herein) as well as causes clearance of the PNA-target gene complex from the cell, thereby reducing potential toxicity.

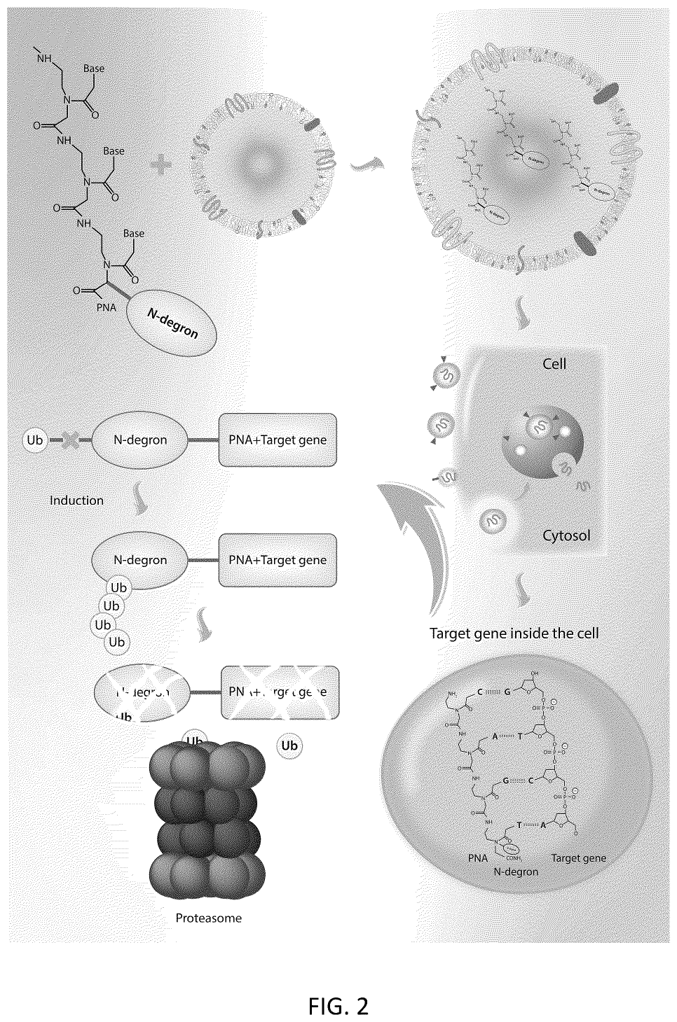

Reference is now made to FIG. 2, which illustratively depicts the operation/use of the herein disclosed PNA-degron platform, according to some embodiment. The operation/use of the complex is similar to that described for FIG. 1, except that the degron is an inducible degron, e.g. a degron which is unrecognizable/cryptic (or other latent) until an inducer is provided/present, e.g., an elevated temperature, hypoxia or a small molecule in the case of a temperature inducible, hypoxia inducible or drug inducible degron, respectively.

Upon administration/presence of the inducer, the degron-PNA-target gene complex is ubiquitinated which in turn bring about its degradation, as essentially described with respect to FIG. 1.

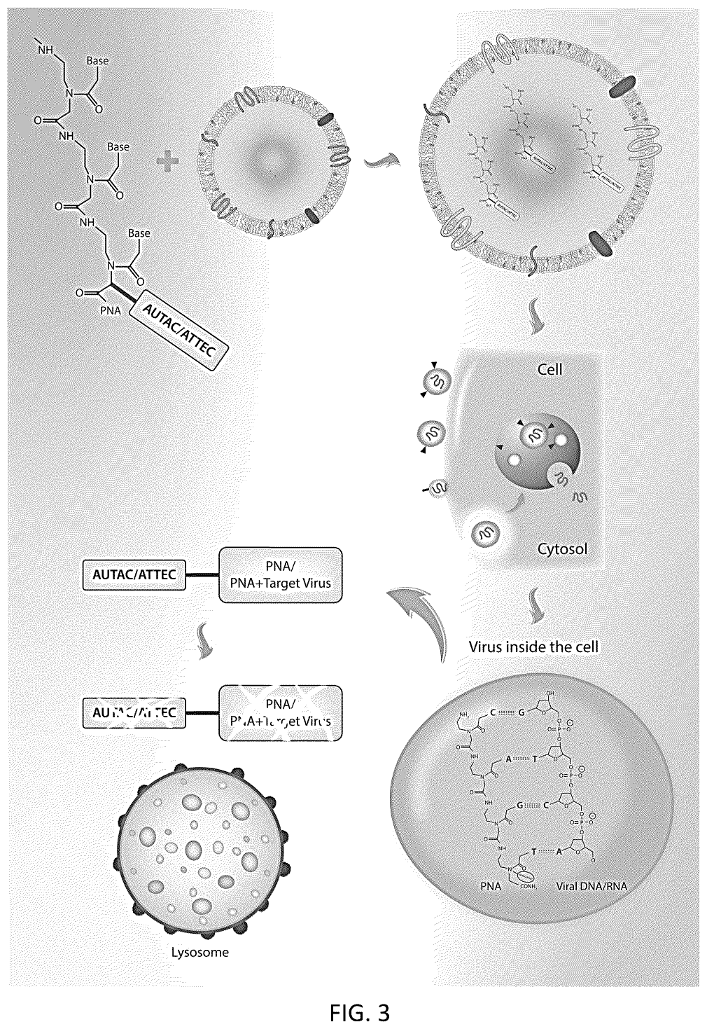

Reference is now made to FIG. 3, which illustratively depicts the operation/use of the herein disclosed PNA-lysosome-targeting-peptide platform, according to some embodiment. Initially, a lysosome-targeting-peptide bound PNA with a nucleobase sequence targeting a gene of interest a viral RNA/DNA (e.g. RNA dependent RNA polymerase of SARS-CoV-2) is provided and assembled within a carrier that facilitates delivery of the lysosome-targeting-peptide bound PNA to the lysosome.

Inside the cell, the lysosome-targeting-peptide bound PNA binds the target gene (here the viral RNA/DNA). Due to the PNA being attached to the lysosome-targeting-peptide, the lysosome-targeting-peptide-PNA-target gene complex enters the lysosome where it is degraded.

Advantageously, this ensures both inhibition of the target gene (as essentially described herein) as well as causes clearance of the PNA-target gene complex from the cell, thereby reducing potential toxicity.

The following examples are presented in order to more fully illustrate some embodiments of the invention. They should in no way be construed, however, as limiting the broad scope of the invention. One skilled in the art can readily devise many variations and modifications of the principles disclosed herein without departing from the scope of the invention.

### EXAMPLES

### Example 1—Synthesizing PNA-Degron Molecule

A PNA degron capable of targeting human coronavirus (OC43) was synthesized according to manufacturer's protocol (Creative Peptides Inc.). The PNA degron synthesized is includes the following interlinked elements:
5-TAMRA (5-Carboxytetramethylrhodamine)—fluorescent dye

(SEQ ID NO: 11)
GCCAATCGCAGATGTTTA
(SEQ ID NO: 1)
Ser-His-Gly-Phe-Pro-Pro-Ala-Val-Ala-Ala-Gln-Asp-
Asp-Gly-Thr-Leu-Pro-Met-Ser-Cys-Ala-Gln-Glu-Ser-
Gly-Met-Asp-Arg-His-Pro-Ala-Ala-Cys-Ala-Ser-Ala-
Arg-Ile-Asn-Val-NH2.

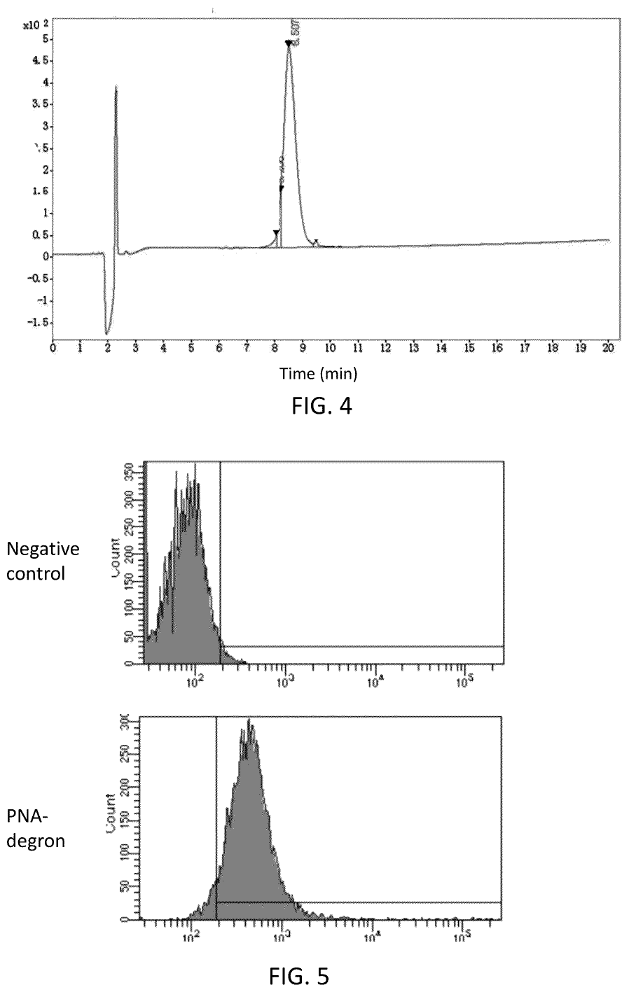

Advantageously, despite its large size a clean PNA-degron molecule was obtained, as seen from the HPLC diagram shown in FIG. 4.

### Example 2—Transfection of Pna-Degron into Human Cells

Lung epithelial cells (HBEpC cells) were plated at a density of 5×10⁵.

The cells were transfected using the silMPORTER transfection system (Merck) according to the manufacturer protocol using 10 μg/ml PNA-degron.

Transfection efficiency was analyzed by evaluation of fluorescence signal by flow cytometry.

As seen from FIG. 5, a significant shift in florescence, indicating that essentially all cells received the PNA-degron molecule (lower panel), as compared to the negative control (transfection of PBS only—lower panel), indicating that the PNA-degron molecule was efficiently transfected into the cells.

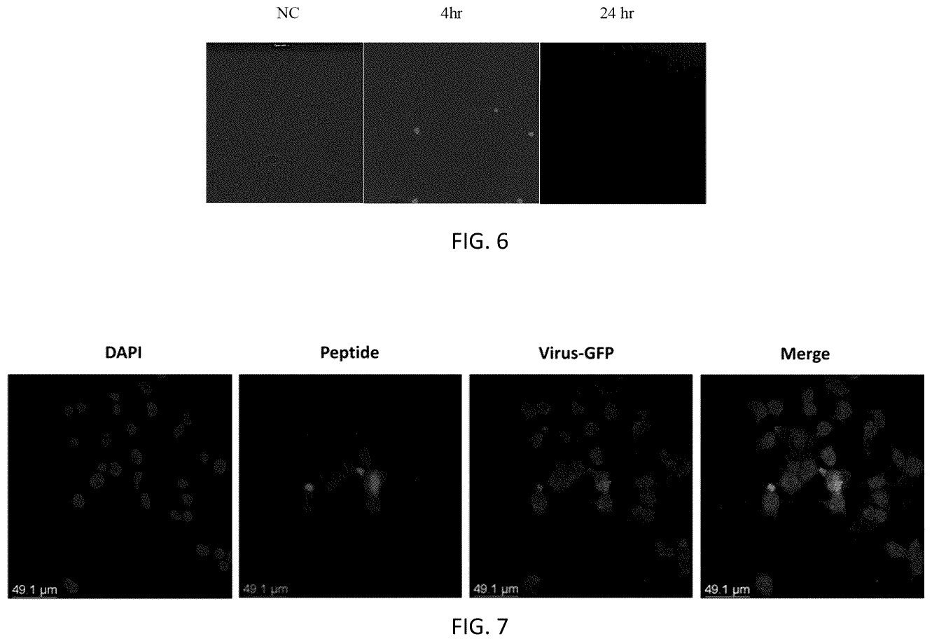

Furthermore, as seen from FIG. 6, while the PNA degron molecule was readily visible in the cells 4 h post transfection, 24 h post transfection no fluorescence was observed. This advantageously demonstrates that the PNA gets degraded and that toxic accumulation of the PNA is avoided.

### Example 3—Effect of PNA-Degron on OC43 Virus

Hek293 cells were transfected with 10 μg/ml of the PNA-degron set forth in Example 1. 2 hours after transfection, the cells were infected with 2MOI of human corona virus (OC43-GFP) or left untreated. Fluorescence was evaluated 4 hours post infection. As seen from FIG. 7, co-localization of the PNA-degron and the virus (GFP tagged) was observed.

Furthermore, as seen from table 1, in untreated cells (no PNA-degron and no viral infection) a mortality of 10% was observed. No additional mortality was observed 24 h post transfection.

As a result of infection with the virus. the mortality of the cells increased to 35.3% of the cells. Advantageously, the mortality rate was reduced by almost 30% when the viral infection was conducted in cells transfected with PNA-degron.

| | Mortality |
|---|---|
| control | 10% |
| OC43 Virus | 35.3% |
| OC43 Virus and PNA-degron | 25% |

These results clearly indicate the ability of the herein disclosed PNA-degron molecule to attenuate the viral infection, with little to no toxicity.

### Example 4—Optimizing PNA Length

### A PNA Degron Molecule

targeting human coronavirus (OC43) was synthesized according to manufacturer's protocol (Creative Peptides Inc.). The PNA-degron molecules synthesized include:
5-TAMRA (5-Carboxytetramethylrhodamine)—fluorescent dye

(SEQ ID NO: 11)
GCCAATCGCAGATGTTTA
(SEQ ID NO: 12)
CAGATTCTTATTATTCTTATATCAT;

and
5-TAMRA (5-Carboxytetramethylrhodamine)—fluorescent dye

(SEQ ID NO: 11)
GCCAATCGCAGATGTTTA
(SEQ ID NO: 13)
CAGATTCTTATTATTCTTAT;

The molecules are transfected into HBEpC, A459, hek293 or HeLa cells, plated at a density of 5×10⁵, using silMPORTER transfection reagent, according to the manufacturer protocol.

Transfection efficiency is then analyzed by flow cytometry and PNA-degron viral-inhibiting efficacy evaluated as in Example 3 and/or by evaluation of viral titer using RT-PCR.

### Example 5—Optimizing PNA-Degron Concentration

HBEpC, A459, hek293 or HeLa cells are plated at a density of 5×10⁵.

The cells are then transfected with different concentrations of PNA-degron (1, 3, 5 and 10 μg/ml) using silMPORTER transfection reagent, according to the manufacturer protocol.

Transfection efficiency is then analyzed by flow cytometry and PNA-degron viral-inhibiting efficacy evaluated as in Example 3 and/or by evaluation of viral titer using RT-PCR.

### Example 6—Optimizing Transfection Agent/Delivery

HBEpC, A459, hek293 or HeLa cells are plated at a density of 5×10⁵.

The cells are then transfected with an optimal concentration of PNA-degron (using various transfection reagents/methods.

Transfection efficiency is then analyzed by flow cytometry and PNA-degron viral-inhibiting efficacy evaluated as in Example 3 and/or by evaluation of viral titer using RT-PCR.

### Example 7—Stability Evaluation

HBEpC, A459, hek293 or HeLa cells are plated at a density of 5×10⁵.

The cells are then transfected with an optimal concentration of PNA-degron (using optimal transfection reagents/methods.

Florescence intensity over time is evaluated to determine PNA-degron degradation by the ubiquitin-protease system (UPS) in the presence and absence of virus.

### Example 8—Viral Load

HBEpC, A459, hek293 or HeLa cells are plated at a density of 5×10⁵.

The cells are then transfected with an optimal concentration of PNA-degron (using optimal transfection reagents/methods.

2 hours after transfection, the cells are infected with 2MOI of human corona virus (OC43-GFP) or left untreated. Fluorescence is evaluated 24 hours and 48 hours post infection.

Viral load (titer) is evaluated using RT-PCR.

### Example 9—Other Viruses

HBEpC, A459, hek293 or HeLa cells are plated at a density of 5×10⁵.

The cells are then transfected with an optimal concentration of PNA-degron molecules specifically targeting various viruses.

2 hours after transfection, the cells are infected with 2MOI of the various viruses or left untreated. Fluorescence is evaluated 24 hours and 48 hours post infection.

Viral load (titer) is evaluated using RT-PCR.

### Example 10—Lysosome Targeting Peptide-PNA Molecule

HBEpC, A459, hek293 or HeLa cells are plated at a density of 5×10⁵.

The cells are then transfected with an optimal concentration of PNA molecules operably linked to a lysosome targeting peptide. Lysosome targeting peptide tested include the AUTAC peptide (SEQ ID NO: 3) and the ATTEC peptide (SEQ ID NO: 4).

2 hours after transfection, the cells are infected with 2MOI of the various viruses or left untreated. Fluorescence is evaluated 24 hours and 48 hours post infection.

Viral load (titer) is evaluated using RT-PCR.

### Example 11—Oncogene

HBEpC, A459, hek293 or HeLa cells are plated at a density of 5×10⁵.

The cells are then transfected with an optimal concentration of PNA-degron molecules specifically targeting oncogenes (e.g. Bc12, c-myc, K-RAS etc.).

Fluorescence is evaluated 24 hours and 48 hours post infection.

Replication rate (cell count) and oncogene expression level in the cells is evaluated 24- and 48-hours post-transfection.

While certain embodiments of the invention have been illustrated and described, it will be clear that the invention is not limited to the embodiments described herein. Numerous modifications, changes, variations, substitutions and equivalents will be apparent to those skilled in the art without departing from the spirit and scope of the present invention as described by the claims, which follow.

1.-23. (canceled)

24. A composition comprising a peptide nucleic acid (PNA) molecule comprising a nucleotide base sequence configured to bind to a target polynucleotide molecule; and a degron protein covalently bound to the PNA, wherein binding of the PNA to the target polynucleotide molecule causes degron mediated degradation of the target polynucleotide molecule.

25. The composition of claim 24, wherein the target polynucleotide molecule is a DNA or an RNA molecule.

26. The composition of claim 24, wherein the target polynucleotide molecule is a viral polynucleotide molecule.

27. The composition of claim 26, wherein the viral polynucleotide molecule is an RNA-dependent RNA polymerase.

28. The composition of claim 24, wherein the target polynucleotide molecule is a human polynucleotide molecule overexpressed in cancer, or a mutated human polynucleotide molecule.

29. The composition of claim 24, wherein the human polynucleotide molecule is Bcl-2, c-myc or KRAS.

30. The composition of claim 24, further comprising a carrier, wherein the carrier is an exosome, a liposome, a nanoparticle or any combination thereof.

31. The composition of claim 24, wherein the degron protein comprises the amino acid sequence set forth in any one of SEQ ID NO: 1-4.

32. The composition of claim 31, wherein the degron protein comprises the amino acid sequence set forth in SEQ ID NO: 1.

33. The composition of claim 24, wherein the nucleobase sequence of the PNA comprises the sequence set forth in SEQ ID NO: 5-10.

34. The composition of claim 24, for use in preventing, ameliorating or treating a viral infection or for use in preventing, ameliorating or treating a cancer in a subject in need thereof.

35. A method for preventing, ameliorating or treating infection of a subject with a virus,
the method comprising administering to the subject a composition comprising a PNA molecule, the PNA molecule comprising:
a nucleotide base sequence configured to bind a polynucleotide of the virus;
and a degron protein covalently bound to the PNA molecule,
wherein binding of the PNA to the viral polynucleotide molecule causes degron mediated degradation of the viral polynucleotide molecule, thereby reducing a load of the virus in the subject.

36. The method of claim 35, wherein the virus is a positive strand RNA virus.

37. The method of claim 36, wherein the virus is a SARS-CoV2 virus.

38. The method of claim 35, wherein the virus is an influenza virus.

39. The method of claim 35, wherein the viral polynucleotide targeted by the PNA is essential to the virus.

40. The method of claim 35, wherein the viral polynucleotide targeted by the PNA molecule is an RNA-dependent RNA polymerase of the virus.

41. A method for preventing, ameliorating or treating cancer in a subject in need thereof,
the method comprising administering to the subject a composition comprising a PNA molecule, the PNA molecule comprising:
a nucleotide base sequence configured to bind a polynucleotide molecule of the subject associated with the cancer; and
a degron protein covalently bound to the PNA molecule,
wherein binding of the PNA molecule to the polynucleotide molecule causes degron mediated degradation of the polynucleotide molecule, thereby reducing expression of the polynucleotide in the subject.

42. The method of claim 41, wherein the polynucleotide targeted by the PNA molecule is a polynucleotide overexpressed in the cancer.

43. The method of claim 41, wherein the polynucleotide targeted by the PNA molecule is a polynucleotide mutated in the cancer.


SEQUENCE LISTING (15 sequences)

SEQ ID NO: 1 - protein, 40 aa, synthetic construct
SHGFPPAVAAQDDGTLPMSCAQESGMDRHPAACASARINV

SEQ ID NO: 2 - protein, 7 aa, synthetic construct
ALAPYIP

SEQ ID NO: 3 - protein, 5 aa, synthetic construct
AUTAC

SEQ ID NO: 4 - protein, 5 aa, synthetic construct
ATTEC

SEQ ID NO: 5 - DNA, 31 bp, synthetic construct
  source 1..31: mol_type = other DNA
  modified_base 1..31: mod_base = OTHER; note = N-2-aminoethyl glycine nucleosides
gccaatcgcagatgtttaccgacgcaaacct

SEQ ID NO: 6 - DNA, 22 bp, synthetic construct
  source 1..22: mol_type = other DNA
  modified_base 1..22: mod_base = OTHER; note = N-2- aminoethyl glycine nucleoside
gatccttatgaagattttcaag

SEQ ID NO: 7 - DNA, 32 bp, synthetic construct
  source 1..32: mol_type = other DNA
  modified_base 1..32: mod_base = OTHER; note = N-2- aminoethyl glycine nucleoside
tagcagattcttattattcttatatcatgcct

SEQ ID NO: 8 - DNA, 17 bp, synthetic construct
  source 1..17: mol_type = other DNA
  modified_base 1..17: mod_base = OTHER; note = N-2- aminoethyl glycine nucleoside
acctcatcaggagatgc

SEQ ID NO: 9 - DNA, 30 bp, synthetic construct
  source 1..30: mol_type = other DNA
acgaggaggagaacttctaccagcagcagc

SEQ ID NO: 10 - DNA, 15 bp, synthetic construct
  source 1..15: mol_type = other DNA
  modified_base 1..15: mod_base = OTHER; note = N-2- aminoethyl glycine nucleoside
cctcccagaggaaaa

SEQ ID NO: 11 - DNA, 18 bp, synthetic construct
  source 1..18: mol_type = other DNA
  modified_base 1..18: mod_base = OTHER; note = N-2- aminoethyl glycine nucleoside
gccaatcgcagatgttta

SEQ ID NO: 12 - DNA, 25 bp, synthetic construct
  source 1..25: mol_type = other DNA
  modified_base 1..25: mod_base = OTHER; note = N-2- aminoethyl glycine nucleoside
cagattcttattattcttatatcat

SEQ ID NO: 13 - DNA, 20 bp, synthetic construct
  source 1..20: mol_type = other DNA
cagattcttattattcttat

SEQ ID NO: 14 - DNA, 21 bp, synthetic construct
  source 1..21: mol_type = other DNA
  modified_base 1..21: mod_base = OTHER; note = N-2- aminoethyl glycine nucleoside
ggacgaggagcggccagagga

SEQ ID NO: 15 - DNA, 24 bp, synthetic construct
  source 1..24: mol_type = other DNA
  modified_base 1..24: mod_base = OTHER; note = N-2- aminoethyl glycine nucleoside
tacatcggcgtcatctacgggggt